(19) United States
(12) Patent Application Publication
(10) Pub. No.: US 2024/0148738 A1
(43) Pub. Date: May 9, 2024

(54) METHOD OF INCREASING THE POPULATION OF BLAUTIA SPP. IN THE GUT MICROBIOME

(71) Applicant: DSM IP Assets B.V., Heerlen (NL)

(72) Inventors: Thanh-Van PHAM, Kaiseraugst (CH); Ateequr REHMAN, Kaiseraugst (CH); Robert STEINERT, Kaiseraugst (CH); Wilbert SYBESMA, Kaiseraugst (CH)

(21) Appl. No.: 18/548,797

(22) PCT Filed: Mar. 3, 2022
(86) PCT No.: PCT/EP2022/055332

§ 371 Date: Sep. 1, 2023

(30) Foreign Application Priority Data
Mar. 5, 2021 (EP) 21160879.9

(51) Int. Cl. A61K 31/525; A61K 31/015; A61K 31/355; A61K 31/375; A61P 1/14

(52) U.S. Cl. CPC A61K 31/525; A61K 31/015; A61K 31/355; A61K 31/375; A61P 1/14

(57) ABSTRACT

The direct delivery of antioxidant compounds (Vitamin B2, Vitamin C, beta-carotene, and Vitamin E) to the large intestine results in an increase in the population of Blautia spp. in the gut. People with lower amounts of Blautia spp. often experience obesity, insulin resistance and chronic inflammation, major depressive disorder as well as other conditions which can be helped by increasing their population of Blautia. An increase in the population of Blautia also increases the amount of beneficial short chain fatty acids in the intestine as well.

### BRIEF DESCRIPTION OF THE INVENTION

The direct delivery of antioxidant compounds (Vitamin B2, Vitamin C, beta-carotene, and Vitamin E) to the large intestine results in an increase in the population of Blautia spp. in the gut. People with lower amounts of Blautia spp. often experience obesity, insulin resistance and chronic inflammation, major depressive disorder as well as other conditions which can be helped by increasing their population of Blautia.

### BACKGROUND OF THE INVENTION

Blautia spp. are commonly found in the gut microbiome. One species, B. wexlerae has been found to be decreased in obese people, those with insulin resistance, and especially in people exhibiting both conditions. Conversely, lean adults have high populations of this microorganism in their microflora. Further, B. wexlerae exerts an anti-inflammatory effect on various blood cells, and thus a higher population can decrease chronic inflammation which is commonly observed in many chronic conditions.

Furthermore, Blautia populations are also seen to be decreased in people suffering from:

Alzheimer's disease; Ankylosing spondylitis, Autism Spectrum Disorder; Atopic dermatitis; Cirrhosis; Colorectal Cancer; Crohn's disease; Graves' disease; HIV; IBD with Clostridium difficile infection; Lung cancer; Major depressive disorder (MDD), Multiple System Atrophy; neuromyelitis optica spectrum disorders (NMOSD; Obesity; Insulin Resistance; Parkinson's disease; Preeclampsia; Schizophrenia; severe acute pancreatitis; Sjögren's syndrome; Type 2 diabetes; ulcerative colitis; visceral fat accumulation. Moreover, increased Blautia was associated with good cognition and decreased systemic inflammation.

An increased population of Blautia spp. can protect against Graft v Host Disease (GVHD), and even increase the survival rate when GVHD occurs.

Blautia wexlerae populations are also seen to be decreased in pregnant women who experience digestive diseases during their pregnancy such as constipation, vomiting and fatty liver.

It would be desirable to be able to increase the population of Blautia spp., and particularly Blautia wexlerae in order to enhance wellness.

### DETAILED DESCRIPTION OF THE INVENTION

It has been found, in accordance with this invention, that the direct delivery of at least one antioxidant selected from the group consisting of Vitamin B2, Vitamin C, beta-carotene, and Vitamin E to the large intestine can increase the population of Blautia spp. in the gut microbiome. The resulting increased Blautia population can assist a person in maintaining a healthy weight, losing weight, maintaining a healthy glycemic balance; prevent or treat hyperglycemia and type 2 diabetes, decrease chronic inflammation, avoid Graft Versus Host Disease, decrease digestive diseases experienced by pregnant women, and who is experiencing, or is at risk of experiencing any of the following conditions:
Alzheimer's disease; unable to maintain healthy cognition;
Ankylosing spondylitis,
Autism Spectrum Disorder;
Atopic dermatitis;
Cirrhosis;
Colorectal Cancer; Lung cancer;
Crohn's disease; ulcerative colitis; Inflammatory Bowel Disease with Clostridium difficile infection;
Graves' disease;
HIV;
Major Depressive Disorder;
Multiple System Atrophy;
neuromyelitis optica spectrum disorders (NMOSD);
Obesity; visceral fat accumulation; unable to maintain a healthy weight;
Insulin Resistance; unable to maintain a healthy glycemic balance; hyperglycemia, Type 2 diabetes;
Parkinson's disease;
Preeclampsia; digestive diseases during pregnancy (including constipation; vomiting; and fatty liver);
Schizophrenia;
severe acute pancreatitis;
Sjögren's syndrome; and
Chronic inflammation.

Thus one aspect of this invention is a method of increasing the population of Blautia spp, and preferably Blautia wexlerae in a person, comprising administering at least one antioxidant selected from the group consisting essentially of: Vitamin B2, Vitamin C, beta-carotene, and Vitamin E directly to the large intestine of the person experiencing, or at risk of experiencing one of the aforementioned conditions, and therefore in need thereof.

Preferably a “person in need thereof” includes a person who is experiencing or is at risk of experiencing at least one of these conditions:
overweight, obese, visceral fat accumulation, or is trying to maintain an ideal weight;
suffering from hyperglycemia, glucose intolerance, unable to maintain a healthy glucose balance; insulin resistance, or Type 2 diabetes
has a condition characterized by chronic inflammation, such as diabetes, dementia, cardiovascular diseases, arthritis and joint diseases, allergies, ankylosing spondylitis, and chronic obstructive pulmonary disease
has or is at risk of developing Graft v Host Disease
has or is at risk of developing cancer, such as colorectal cancer and lung cancer
is pregnant and experiences a digestive disease (vomiting, constipation, or Fatty Liver disease) associated with the pregnancy; or is pregnant and wishes to avoid such a digestive disease, or is at risk of preeclampsia;
Intestinal conditions or diseases such as: Crohn's disease; ulcerative colitis; Inflammatory Bowel Disease with Clostridium difficile infection;
Autoimmune diseases such as: Sjögren's syndrome, Graves' disease
Experiencing issues involving brain health and/or mental wellness, such as Major Depressive disorder, dementia, autism spectrum disorder, Parkinson's disease, schizophrenia, Alzheimer's disease, neuromyelitis optica spectrum disorders (NMOSD), or neurodegenerative disorder such as multiple system atrophy; and
Other conditions such as atopic dermatitis, cirrhosis, HIV, severe acute pancreatitis.

### BRIEF DESCRIPTION OF THE FIGURES

FIG. 1 shows the modified version of a continuous batch fermentation model (such as the SHIME, or TWINSHIME, or QuadSHIME provided by Prodigest, Technologiepark-Zwijnaarde 94, 9052 Gent, Belgium), which were used for the current study. St: Stomach vessel, SI: Small Intestine vessel, St/SI: vessel serving as stomach and small intestine, PC: Proximal colon and DC: Distal colon.

FIG. 2. Effect of the antioxidant blend (AOB) and the prebiotic XOS treatments compared to a blank control (CTRL) on the abundance of Blautia wexlerae in the proximal (A) and distal colon (B). * significant difference compared to the blank control (p<0.05).

### DEFINITIONS

As used throughout, the following definitions apply:

The term “riboflavin” which can be used interchangeably with “Vitamin B2”, includes riboflavin and esters thereof, in particular riboflavin-5′-phosphate.

The term “vitamin C” which can be used interchangeably with “ascorbic acid” also includes pharmaceutically acceptable salts thereof (e.g. sodium ascorbate and calcium ascorbate) and pharmaceutically acceptable esters thereof (in particular ascorbyl palmitate).

The term “β-carotene” refers to β-carotene or Provitamin A.

The term “vitamin E” includes four forms of tocopherols (alpha-Tocopherol, beta-Tocopherol, gamma-Tocopherol and delta-Tocopherol) and four forms of tocotrienols (alpha-tocotrienols, beta-tocotrienols, gamma-tocotrienols and delta-tocopherols.

The term “directly delivered” means that the antioxidant(s) are formulated such that they are available to the lower intestine gut microflora. There are a variety of delayed-release or sustained-release forms which can accomplish this known in the art.

The term “treating” includes therapeutically treating or non-therapeutically treating.

The term “AOB” means antioxidant blend, which is the combination of riboflavin, Vitamin C, Beta-carotene, and Vitamin E.

Conditions Related to Excess Weight

The gut microbiome influences how food is metabolized, and a high population of Blautia wexlerae is associated with a lean body mass. Thus a person's weight loss can be enhanced with the addition of at least one directly-delivered antioxidant. Preferably all four antioxidants are directly delivered to the lower gut (Vitamin B2, Vitamin C, beta-carotene, and Vitamin E). The antioxidants also assist in weight maintenance, weight maintenance after weight loss, and weight gain prevention (without a previous weight loss). Preferably the antioxidants are taken in addition to a diet intended for weight loss and/or increasing exercise.

Thus, one embodiment of this invention is a method of losing weight, maintaining weight and/or preventing or ameliorating weight gain comprising administering at least one antioxidant selected from the group consisting of Vitamin B2, Vitamin C, beta-carotene, and Vitamin E to an individual who is obese, overweight, or wishes to maintain healthy body weight. Another embodiment is the use of at least one directly delivered antioxidant selected from the group consisting of: Vitamin B2, Vitamin C, beta-carotene, and Vitamin E for treating or preventing obesity; reducing body weight, maintaining body weight, ameliorating body weight gain. Another embodiment is the use of at least one antioxidant selected from the group consisting of Vitamin B2, Vitamin C, beta-carotene, and Vitamin E in the manufacture of a medicament or nutraceutical to lose bodyweight, maintain body weight or ameliorate body weight gain. In each of these embodiments, it is preferred that all four antioxidants are delivered.

Conditions Related to Glycemic Control

Persons who suffer from hyperglycemia and type 2 diabetes, or other conditions relating to blood sugar regulation have been observed to have fewer Blautia in their gut microbiome. While not wishing to be bound by theory, this may be related to the utilization of carbohydrates in the diet and Blautia's influence thereon.

Thus, another embodiment of this invention is a method of normalizing increased blood sugar levels, lessening hyperglycemia, treating, ameliorating, or preventing type 2 diabetes comprising administering directly to the large intestine a composition comprising at least one antioxidant selected from the group consisting of Vitamin B2, Vitamin C, beta-carotene, and Vitamin E, to a person in need thereof. Another embodiment is the use of a composition comprising at least one antioxidant selected from the group consisting of Vitamin B2, Vitamin C, beta-carotene, and Vitamin E for normalizing increased blood sugar levels, lessening hyperglycemia, treating, ameliorating, or preventing type 2 diabetes, wherein the composition is formulated for direct delivery to the large intestine. Another embodiment is the use of a composition comprising at least one antioxidant selected from the group consisting of: Vitamin B2, Vitamin C, beta-carotene, and Vitamin E, in the manufacture of a medicament for normalizing increased blood sugar levels, lessening hyperglycemia, treating, ameliorating, or preventing type 2 diabetes. Preferably, in all of the above embodiments, at least two antioxidants are present. In more preferred embodiments the composition comprises Vitamin B2, Vitamin C, beta-carotene, and Vitamin E.

Preeclampsia and Digestive Diseases During Pregnancy

A decreased level of Blautia has been observed in pregnant women who suffer from preeclampsia or vomiting, constipation, and/or or fatty liver disease (hereinafter “digestive disease” related to pregnancy). Increasing the levels of Blautia will prevent, ameliorate, or treat these conditions. Thus one embodiment of this invention is a method of preventing, treating, or ameliorating the severity of, preeclampsia, or a digestive disease experienced by a pregnant woman comprising administering a composition comprising at least one antioxidant selected from the group consisting of: Vitamin B2, Vitamin C, beta-carotene, and Vitamin E, formulated for direct delivery to the large intestine of the pregnant woman. Another embodiment is the use of a composition comprising at least one antioxidant selected from the group consisting of: Vitamin B2, Vitamin C, beta-carotene, and Vitamin E, formulated for direct delivery to the large intestine, for the prevention, reducing the severity of, or treatment of preeclampsia or a digestive disease in a pregnant woman. Another embodiment is the use of a composition comprising at least one antioxidant selected from the group consisting of Vitamin B2, Vitamin C, beta-carotene, and Vitamin E, in the manufacture of a medicament for preventing, ameliorate the severity of, or treating preeclampsia or a digestive disease experienced by a pregnant woman.

Preferably, in all of the above embodiments, at least two antioxidants are present. In more preferred embodiments, the composition comprises Vitamin B2, Vitamin C, beta-carotene, and Vitamin E.

Chronic Inflammation

Chronic inflammation is a symptom of a number of diseases, including diabetes, dementia, cardiovascular diseases, arthritis and joint diseases, allergies, and chronic obstructive pulmonary disease; hereinafter referred to as (hereinafter referred to as “chronic inflammatory disease”). It has been observed that Blautia populations are decreased in all of these, and thus an increase in Blautia will prevent, lessen, or ameliorate the severity of symptoms, or treat the symptoms of the aforementioned diseases.

Thus, another embodiment of this invention is a method of preventing, lessening or ameliorating the severity of symptoms, or treating the symptoms of a chronic inflammatory disease comprising administering directly to the large intestine a composition comprising at least one antioxidant selected from the group consisting of Vitamin B2, Vitamin C, beta-carotene, and Vitamin E, to a person in need thereof. Another embodiment is the use of a composition comprising at least one antioxidant selected from the group consisting of Vitamin B2, Vitamin C, beta-carotene, and Vitamin E wherein the composition is formulated for direct delivery to the large intestine for use in preventing, lessening or ameliorating the severity of symptoms or treating the symptoms of chronic inflammatory disease. Another embodiment is the use of a composition comprising at least one antioxidant selected from the group consisting of: Vitamin B2, Vitamin C, beta-carotene, and Vitamin E, in the manufacture of a medicament for preventing, lessening or ameliorating the severity of symptoms, or treat the symptoms of chronic inflammatory disease. Preferably, in all of the above embodiments, at least two antioxidants are present. In more preferred embodiments, the composition comprises Vitamin B2, Vitamin C, beta-carotene and Vitamin E.

Intestinal Related Conditions

“Intestinal Related Conditions” include intestinal condition or diseases such as: Crohn's disease; ulcerative colitis; Inflammatory Bowel Disease with Clostridium difficile infection (“IBD”) each of which are characterized by a decrease in the Blautia population. Thus an increase in Blautia would treat, prevent, or ameliorate adverse symptoms of these diseases. One embodiment of this invention is a method of preventing, lessening or ameliorating the severity of symptoms, or treating the symptoms of an intestinal condition selected from the group consisting of Crohn's disease, ulcerative colitis, and IBD, comprising administering directly to the large intestine a composition comprising at least one antioxidant selected from the group consisting of Vitamin B2, Vitamin C, beta-carotene, and Vitamin E, to a person in need thereof. Another embodiment is the use of a composition comprising at least one antioxidant selected from the group consisting of Vitamin B2, Vitamin C, beta-carotene, and Vitamin E wherein the composition is formulated for direct delivery to the large intestine, for use in preventing, lessening or ameliorating the severity of symptoms, or treating the symptoms of an intestinal condition selected from the group consisting of Crohn's disease, ulcerative colitis, and IBD. Another embodiment is the use of a composition comprising at least one antioxidant selected from the group consisting of: Vitamin B2, Vitamin C, beta-carotene, and Vitamin E, in the manufacture of a medicament for preventing, lessening or ameliorating the severity of symptoms, or treat the symptoms of an intestinal condition selected from the group consisting of Crohn's disease, ulcerative colitis, and IBD. Preferably, in all of the above embodiments, at least two antioxidants are present. In more preferred embodiments the composition comprises Vitamin B2, Vitamin C, beta-carotene, and Vitamin E.

Conditions Relating to Brain Health or Mental Wellness

A decreased population of Blautia has also been associated with various mental illnesses and/or brain disorders. Further, a higher population has been positively associated with cognition. Thus, one embodiment of this invention is a method of preventing, lessening or ameliorating the severity of symptoms, or treating the symptoms of a brain disease or mental illness selected from the group consisting of: major depressive disorder, dementia, autism spectrum disorder, Parkinson's disease, schizophrenia, Alzheimer's disease, neuromyelitis optica spectrum disorders and Multiple system atrophy, comprising administering directly to the large intestine a composition comprising at least one antioxidant selected from the group consisting of Vitamin B2, Vitamin C, beta-carotene, and Vitamin E, to a person in need thereof. Another embodiment is the use of a composition comprising at least one antioxidant selected from the group consisting of Vitamin B2, Vitamin C, beta-carotene, and Vitamin E wherein the composition is formulated for direct delivery to the large intestine, for use in preventing, lessening or ameliorating the severity of symptoms, or treating the symptoms of a condition related to brain disease or mental health selected from the group consisting of: major depressive disorder, dementia, autism spectrum disorder, Parkinson's disease, schizophrenia, Alzheimer's disease, neuromyelitis optica spectrum disorders and Multiple system atrophy. Another embodiment is the use of a composition comprising at least one antioxidant selected from the group consisting of: Vitamin B2, Vitamin C, beta-carotene, and Vitamin E, in the manufacture of a medicament for preventing, lessening or ameliorating the severity of symptoms, or treat the symptoms of a brain disease or mental illness selected from the group consisting of: major depressive disorder, dementia, autism spectrum disorder, Parkinson's disease, schizophrenia, Alzheimer's disease, neuromyelitis optica spectrum disorders and Multiple system atrophy. Preferably, in all of the above embodiments, at least two antioxidants are present. In more preferred embodiments the composition comprises Vitamin B2, Vitamin C, beta-carotene, and Vitamin E.

Graft v Host Disease

A decreased population of Blautia has been observed in people suffering from Graft v Host disease. Thus, increasing the population of Blautia can be helpful in treating or lessening the symptoms associated with Graft v Host disease. Thus, one embodiment of this invention is a method of preventing, lessening or ameliorating the severity of symptoms, or treating the symptoms of Graft V Host Disease, comprising administering directly to the large intestine a composition comprising at least one antioxidant selected from the group consisting of Vitamin B2, Vitamin C, beta-carotene, and Vitamin E, to a person in need thereof. Another embodiment is the use of a composition comprising at least one antioxidant selected from the group consisting of Vitamin B2, Vitamin C, beta-carotene, and Vitamin E wherein the composition is formulated for direct delivery to the large intestine, for use in preventing, lessening or ameliorating the severity of symptoms, or treating the symptoms of Graft V Host Disease. Another embodiment is the use of a composition comprising at least one antioxidant selected from the group consisting of: Vitamin B2, Vitamin C, beta-carotene, and Vitamin E, in the manufacture of a medicament for preventing, lessening or ameliorating the severity of symptoms, or treat the symptoms of Graft v Host disease. Preferably, in all of the above embodiments, at least two antioxidants are present. In more preferred embodiments the composition comprises Vitamin B2, Vitamin C, beta-carotene, and Vitamin E.

Increasing Short Chain Fatty Acid Production

It has also been found, in accordance with this invention, that the amount of short chain fatty acids (SCFAs) present in the large intestine can be increased by administration of the antioxidants. Blautia is a species which produces SCFAs, and in particular formic acid, acetic acid, propionic acid, butyric acid, 2-methylpropanoic acid, 3-methylbutanoic acid, and hexanoic acid. The most important SCFAs are acetic acid, propionic acid and butyric acid. Thus, increasing Blautia is a way of increasing the beneficial effects of increasing SCFAs, which include generally improving intestinal health, increasing the activity of the gut flora, and decreasing the abundance of pathogens.

Thus another embodiment of this invention is a method of increasing the amount of at least one SCFA, selected from the group consisting of: formic acid, acetic acid, propionic acid, butyric acid, 2-methylpropanoic acid, 3-methylbutanoic acid, and hexanoic acid comprising increasing the population of Blautia spp in the microbial flora.

Doses:

In all cases, the directly delivered antioxidant(s) can be used as the sole therapy, or can be combined with additional medicaments, or modified behaviors to enhance efficacy.

Preferably, Vitamin B2 can be administered in an amount such that its local concentration in the colon is at least 0.01 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. Specific dosages per day can range up to 200 mg/day, preferably 5-100 mg/day, more preferably from 10-50 mg/day.

Preferably, β-carotene is administered in an amount such that its local concentration in the colon is at least 0.1 g/L, preferably at least 0.15 g/L, most preferably at least 0.2 g/L. Preferred local concentrations in the colon range from about 0.05 g/L to about 0.4 g/L, more preferably from about 0.15 g/L to about 0.25 g/L One preferred dosage per day is up to 150 mg.

Preferably, vitamin E (50%) is administered in an amount such that its local concentration in the colon is at least 0.005 g/L preferably at least 0.05 g/L, most preferably at least 0.15 g/L. Preferred local concentrations in the colon range from about 0.005 g/L to about 2.5 g/L, more preferably from about 0.15 g/L to about 1.75 g/L. One preferred dosage per day is up to 1000 mg.

Preferably, Ascorbic Acid can be administered in an amount such that its local concentration in the colon is at least 0.05 g/L, preferably at least 0.1 g/L, most preferably at least 2 g/L. Preferred local concentrations in the colon range from about 0.05 g/L to about 1.5 g/L, more preferably from about 0.5 g/L to about 1 g/L, most preferably from about 0.8 g/L to about 0.9 g/L. Specific dosages per day can range up to 2000 mg/day, preferably 100-2000 mg/day; more preferably 200-1000 mg/day.

Preferably the antioxidants are present in a ratio of:

| Riboflavin | 0.5 to 2 |
|---|---|
| Ascorbic Acid | 4 to 15 |
| Vitamin E | 1 to 5 |
| Beta-Carotene | 0.5-3 |

More preferably the ratio of Riboflavin/Ascorbic acid/Vitamin E/β-Carotene is 1.0/6.6/1.3/1.6.

In preferred embodiments, the compositions are administered for an extended period time, such as for at least once per day for at least 3 days, at least a week, at least two weeks and at least 4 weeks.

The antioxidants are preferably administered in a formulation which allows the antioxidants to be released in the large intestine. Such forms are generally known in the art. Alternatively, and perhaps preferably for non-human administration, the animal is administered a high enough dose for the antioxidant to overcome absorption in the upper intestine and be present in the large intestines.

The following non-limiting Examples are presented to better illustrate the invention

### Examples

The aim of this study was to compare the effect of directly delivered antioxidants to that of two established prebiotics: Xylooligosacchrides (XOS). The in vitro experiments presented below are designed to mimic the in vivo situation where the antioxidant(s) are formulated such that they are available to the lower intestine gut microflora.

A donor was selected for the long-term SHIME® experiment, where the impact of repeated intake of the test products was evaluated on the composition (as assessed via 16S Illumina sequencing) of the luminal gut microbiome.

Design of the SHIME® Experiment

The typical reactor setup of the SHIME® represents the gastrointestinal tract of the adult human. It has a succession of five reactors simulating the different parts of the human gastrointestinal tract. The first two reactors are of the fill-and-draw principle to simulate different steps in food uptake and digestion, with peristaltic pumps adding a defined amount of SHIME feed (140 mL 3×/day) and pancreatic and bile liquid (60 mL 3×/day), respectively to the stomach (V1) and small intestine (V2) compartment and emptying the respective reactors after specified intervals. The last three compartments simulate the large intestine. These reactors are continuously stirred; they have a constant volume and pH control. Retention time and pH of the different vessels are chosen to resemble in vivo conditions in the different parts of the colon. Upon inoculation with fecal microbiota, these reactors simulate the ascending (V3), transverse (V4) and descending (V5) colon. Inoculum preparation, retention time, pH, temperature settings and reactor feed composition have been described elsewhere. Upon stabilization of the microbial community in the different regions of the colon, a representative microbial community is established in the three colon compartments, which differs both in composition and functionality in the different colon regions.

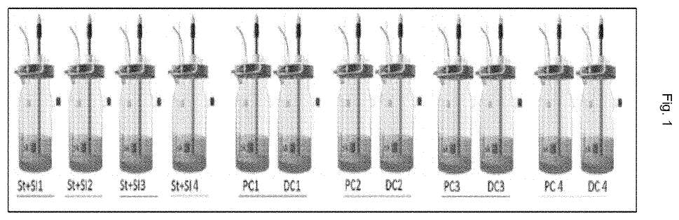

The conventional SHIME setup was adapted from a TWINSHIME configuration to a QuadSHIME® configuration (FIG. 1) allowing us to compare four different conditions in parallel. During this specific project, the properties of three different test ingredients and a blank control were evaluated in a parallel TripleSHIME® configuration using the microbiota of a healthy adult human donor. As a compromise for the additional test conditions, the colon regions were limited to two regions as compared to three regions in the TWINSHIME. The retention times and pH ranges were optimized in order to obtain results that are representative of a full GIT simulation. In practice, in QuadSHIME® experiments, instead of working with 2 units, each composed of an AC-TC-DC configuration (ascending, transverse and descending colon), one used 4 PC-DC units. Upon inoculation with a faecal microbiota of a human adult, these reactors simulate the proximal colon (PC; pH 5.6-5.9; retention time=20 h; volume of 500 mL) and distal colon (DC; pH 6.6-6.9; retention time=32 h; volume of 800 mL).

The SHIME® experiment for this study consisted of two stages (Table 1, below):

Stabilization period: After the inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the fecal inoculum. Analysis of samples at the end of this period allows to determine the baseline microbial community composition and activity in the different reactors.

Treatment period: During this two-week period, the SHIME reactor was operated under nominal conditions, but with a diet supplemented with the test product. Samples taken from the colon reactors in this period allow to investigate the specific effect on the resident microbial community composition and activity. For the blank control condition, the standard SHIME nutrient matrix was further dosed to the model for a period of 14 days. Analysis of samples of these reactors allow to determine the nominal microbial community composition and activity in the different reactors, which will be used as a reference for evaluating the treatment effects.

TABLE 1
Overview of the different stages applied in this study.

| Week 1 | Week 2 | Week 3 | Week 4 |
|---|---|---|---|
| Stabilization | Stabilization | Treatment | Treatment |

Samples were collected at the following time points to follow up on the adaptation of the microbiota to the different test products:
Last three days of stabilization period;
Last two days of the first treatment week;
Last two days of the second treatment week.

Analysis of the Microbial Community Composition and Activity

An important characteristic of the SHIME is the possibility to work with a stabilized microbiota community and to regularly collect samples from the different intestinal regions for further analysis. The large volumes in the colonic regions allow to collect sufficient volumes of liquids each day, without disturbing the microbial community or endangering the rest of the experiment. A number of microbial parameters are monitored throughout the entire SHIME experiment. These measurements are necessary to evaluate the performance of the model and allow to monitor basic changes in the microbial community composition and activity due to the prebiotic treatment.

Microbial Community Composition:

Samples were collected for 16S rRNA gene-targeted Illumina sequencing.

Analysis of the Microbial Community Composition

Two techniques were combined to map the community shifts induced by the different treatments in considerable detail:
16S rRNA gene-targeted Illumina sequencing, a PCR-based method by which microbial sequences are amplified until saturation, thus providing proportional abundances of different taxa at different phylogenetic levels (microbial phylum, family and OTU level). The methodology applied by ProDigest involves primers that span 2 hypervariable regions (V3-V4) of the 16S rDNA. Using a paired sequencing approach, sequencing of 2×250 bp results in 424 bp amplicons. Such fragments are taxonomically more useful as compared to smaller fragments that are taxonomically less informative.
Accurate quantification of total bacterial cells in the samples through flow cytometry. Combining the high-resolution phylogenetic information of the 16S rRNA gene-targeted Illumina together with the accurate enumeration of the cell count via flow cytometry, highly accurate, quantitative abundances of the different taxonomic entities inside the reactors can be obtained.

The comparisons of normally distributed data of the different stabilization and treatment weeks on microbial metabolic markers and microbial community parameters were performed with a Student's T-test assuming equal variance. Differences were considered significant if p<0.05.

Trial

Three different test products were tested in this project as compared to a blank control. The test products and the in vitro doses at which they were tested can be found in Table 2.

TABLE 2
List of test products and the in vitro dosage at which
they were tested in the long-term SHIME experiment.

| | | | In vitro |
|---|---|---|---|
| | | Product | dosage (mg/d) |
| Blend 1 | Vitamin | Riboflavin | 75 |
| | antioxidant | Ascorbic acid | 500 |
| | mix (AOB) | Dry Vitamin E (50% form) | 200 |
| | | β-Carotene (10% form) | 1200 |
| Blend 2 | Prebiotic | XOS | 3000 |

Results

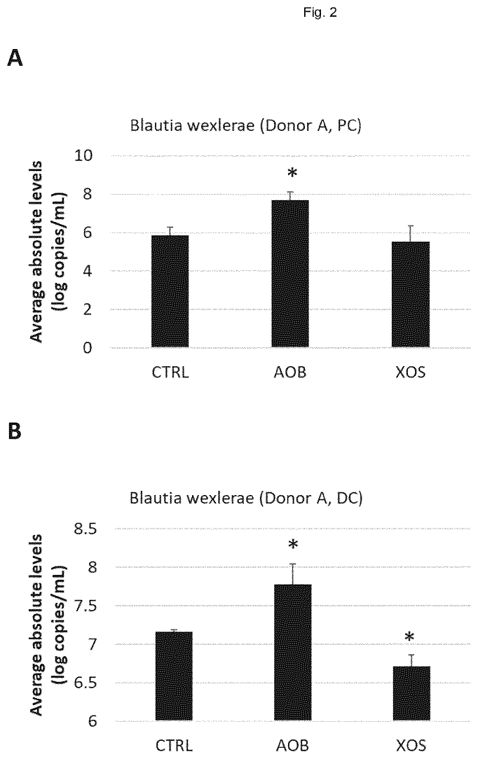

Increased Abundance of Beneficial Bacteria Blautia wexlerae (FIG. 2)

Treatment of proximal and distal colon vessels with antioxidant resulted in significantly higher Blautia wexlerae abundance if compared to control. Interestingly, this increase was even more than the prebiotic XOS treated vessels.

1. A method of increasing the population of Blautia sp. in the gut microbiome comprising directly delivering to the large intestine at least one antioxidant selected from the group consisting of Vitamin B2, Vitamin C, beta-carotene, and Vitamin E.

2. A method according to claim 1, wherein the Blautia wexlerae population is increased.

3. A method according to claim 1, wherein the population of Blautia population was decreased prior to direct delivery of the at least one antioxidant.

4. A method according to claim 1, wherein a person is experiencing or is at risk of experiencing at least one of the following conditions:
Alzheimer's disease; unable to maintain healthy cognition; Ankylosing spondylitis, Autism Spectrum Disorder; Atopic dermatitis; Cirrhosis; Colorectal Cancer; Lung cancer; Crohn's disease; ulcerative colitis; Inflammatory Bowel Disease with Clostridium difficile infection; Graves' disease; HIV; Major Depressive Disorder; Multiple System Atrophy; neuromyelitis optica spectrum disorders (NMOSD); Obesity; visceral fat accumulation; unable to maintain a healthy weight; Insulin Resistance; unable to maintain a healthy glycemic balance; hyperglycemia, Type 2 diabetes; Parkinson's disease; Preeclampsia; digestive diseases during pregnancy (including constipation; vomiting; and fatty liver); Schizophrenia; severe acute pancreatitis; Sjögren's syndrome; and Chronic inflammation.

5. A method according to claim 1, wherein a short chain fatty acid in the intestine is increased.

6. A method according to claim 1, wherein Vitamin B2, Vitamin C, beta-carotene, and Vitamin E are delivered directly to the gut.

7. Use of at least one antioxidant selected from the group consisting of Vitamin B2, Vitamin C, beta-carotene, and Vitamin E directly delivered to the large intestine to increase the population of Blautia sp. in the gut microbiome.

8. Use according to claim 7, wherein the population of Blautia wexlerae population is increased.

9. Use according to claim 7, wherein the population of Blautia was decreased prior to the use of the at least one directly delivered antioxidant.

10. Use according to claim 7, wherein a person is experiencing or is at risk of experiencing at least one of the following conditions:
Alzheimer's disease; unable to maintain healthy cognition; Ankylosing spondylitis, Autism Spectrum Disorder; Atopic dermatitis; Cirrhosis; Colorectal Cancer; Lung cancer; Crohn's disease; ulcerative colitis; Inflammatory Bowel Disease with Clostridium difficile infection; Graves' disease; HIV; Major Depressive Disorder; Multiple System Atrophy; neuromyelitis optica spectrum disorders (NMOSD); Obesity; visceral fat accumulation; unable to maintain a healthy weight; Insulin Resistance; unable to maintain a healthy glycemic balance; hyperglycemia, Type 2 diabetes; Parkinson's disease; Preeclampsia; digestive diseases during pregnancy (including constipation; vomiting; and fatty liver); Schizophrenia; severe acute pancreatitis; Sjögren's syndrome; and Chronic inflammation.

11. Use according to claim 7, wherein a short chain fatty acid in the intestine is increased.

12. Use according to claim 7, wherein Vitamin B2, Vitamin C, beta-carotene, and Vitamin E are delivered directly to the gut.
